(19) United States
(12) Patent Application Publication
(10) Pub. No.: US 2012/0130143 A1
(43) Pub. Date: May 24, 2012

(54) PROCESS FORTHE CONVERSION OF A HYDROCARBONACEOUS FEESTOCK

(76) Inventor: Edmundo Steven Van Doesburg, Amsterdam (NL)

(21) Appl. No.: 13/383,887

(22) PCT Filed: Jul. 15, 2010
(86) PCT No.: PCT/EP2010/060189

§ 371 Date: Feb. 7, 2012

(30) Foreign Application Priority Data
Jul. 15, 2009 (EP) 09165522.5

(51) Int. Cl. C07C 7/163

(52) U.S. Cl. 585/836; 585/841

(57) ABSTRACT

A hydrocarbon oil is hydrotreated in a process employing at least a first and a second reactor vessel, which process comprises: (i) contacting the hydrocarbon oil in the first reactor vessel at elevated temperature and pressure with a hydrotreating catalyst in the presence of a hydrogen-containing gas, thereby consuming hydrogen; (ii) separating the effluent of step (i) into partly hydrotreated hydrocarbon oil and contaminated hydrogen containing gas using a stripping column employing used hydrogen-containing gas as stripping gas; (iii) contacting partly hydrotreated hydrocarbon oil obtained in step (ii) in the second reactor vessel at elevated temperature and pressure with a hydrotreating catalyst in the presence of clean hydrogen-containing gas, thereby consuming hydrogen, wherein at least 80% of the hydrogen consumed in steps (i) and (iii) is supplemented by additional clean hydrogen-containing gas fed to the second reactor; (iv) separating the product from step (iii) in the second reactor vessel into a hydrotreated hydrocarbon oil and used hydrogen-containing gas, which hydrotreated hydrocarbon oil can be recovered as product and, (v) transporting at least a portion of used hydrogen-containing gas obtained in step (iv) to step (ii) for use as stripping gas.

The present invention relates to a process for hydrotreating a hydrocarbon oil employing at least a first and a second reactor vessel in series.

Processes for hydrotreating hydrocarbon oils are well known. Also processes that employ two or more reactors have been described in the literature.

Processes for reducing the amount of sulphur or nitrogen containing compounds and aromatics, are in general called hydrotreating processes. These processes can be further divided into processes which are especially directed at saturation of unsaturated compounds such as aromatics and olefins, in which case they are called hydrogenation processes, and processes which are especially directed at reducing the amount of sulphur containing compounds and often at the same time also of nitrogen containing compounds, in which case they are called hydrodesulphurisation processes. There are also processes which are specifically directed at reducing the amount of nitrogen containing compounds and in which only a relatively small amount of sulphur-containing compounds are removed. These are called hydrodenitrogenation processes. With the expression hydrodesulphurisation processes, which is used hereinafter, processes are meant which are directed at removal of sulphur-containing compounds and optionally an amount of nitrogen. Processes wherein linear waxy hydrocarbons are isomerised to branched alkanes are referred to as hydroisomerisation or as hydrodewaxing processes. These processes can be applied to middle distillates so that the pour point is reduced. Alternatively, the process can be applied to lubricating oils to enhance the viscosity index.

A hydrotreating process using two reactors in series has been described in EP-A 611 816. It describes a hydrotreating process in which process a fresh hydrocarbon oil in a first reactor vessel is contacted with a hydrotreating catalyst in the presence of used hydrogen-containing gas. The effluent of this contact is separated into partly hydrotreated hydrocarbon oil and contaminated hydrogen-containing gas. The separation is carried out in a stripping column using fresh clean hydrogen as stripping gas. The partly hydrotreated hydrocarbon oil is contacted in a second reactor vessel with a hydrotreating catalyst in the presence of clean hydrogen containing gas. The product of this step is separated into a hydrotreated hydrocarbon oil and used hydrogen-containing gas, which hydrotreated hydrocarbon oil can be recovered as product, and which used hydrogen-containing gas is passed to the first reactor vessel.

The process according to this prior art has the drawback that although it has conducted some integration to enhance the economics of the process, it uses clean hydrogen-containing gas in the stripping column. Therefore, the clean gas is thereby mixed with contaminants from the effluent of the first hydrotreating reactor. Since the resulting stream contains significant amounts of hydrogen sulphide, it is cleaned, e.g., by an amine treatment, before it is re-used in the hydrotreatment reactors. It is evident that in this way the clean hydrogen-containing gas stream is unnecessarily contaminated and subsequently cleaned, without having been used in a hydrotreatment reaction. Further, the cold clean hydrogen-containing stripping gas will cause considerable cooling of the partly hydrotreated hydrocarbon oil, thereby increasing the heat requirement to achieve the desired hydrotreating conditions for the partly hydrotreated oil in the second reactor.

A hydroprocessing process with integrated interstage stripping has been described in WO-A-200248285. It describes a hydroprocessing process wherein interstage stripping is provided between two hydroprocessing zones. The effluent from the second reactor is passed through a heat exchanger where it is cooled, to a separation zone. The separator in the separation zone separates liquid product from the gases. The gaseous products from the separator are passed to the first separator, where it is used as stripping gas. Some of the gaseous product may be sent to the feed to the first reactor.

The disadvantage of the described process is that the cooled recycled gas will cause considerable cooling of the partly hydrotreated hydrocarbon oil in the stripper, thereby increasing the heat requirement to achieve the desired hydrotreating conditions for the partly hydrotreated oil in the second reactor. A further disadvantage is that the main supply of hydrogen is to the first reactor. The fresh hydrogen is unnecessarily contaminated.

In US-A-20060118466 a hydrotreatment process is described comprising at least two reaction steps with intermediate fractionation. The stripper is being operated with purified make-up hydrogen. Furthermore, the effluent from the second reactor is cooled, and the gases are separated using a drum separator at low temperatures. Also here the hydrogen containing gas is being cooled before being re-used.

In WO-A-2003054118 a method is being described to produce a lube basestock. The hydrogen containing recycle gas is being cleaned before being re-used. The cold clean hydrogen-containing gas will cause considerable cooling of the partly hydrotreated hydrocarbon oil, thereby increasing the heat requirement to achieve the desired hydrodewaxing conditions oil in the second reactor. Also in U.S. Pat. No. 2,671,754 the hydrogen from the second reactor is being cooled, followed by a separate separation step, before being recycled. The stripper between the two reactors is being fed with substantially sulphur free hydrogen.

A hydrocracking process for the production of high quality distillates from heavy gas oils is being described in WO-A-2003080769. The invention is directed to the use of hot strippers and separators between the first and second reactor stages, employing a single hydrogen loop. The stream coming from the top of the second reactor, containing primarily hydrogen, is cooled by a heat exchanger and send to cold high pressure separator. Also here the hydrogen containing gas is being cooled before being re-used. A further disadvantage of the cooled recycle gas is that it requires recompression before it can be re-used in the stripper or reactor.

The present invention aims at further optimising the process of the prior art.

Accordingly, the present invention provides a process for hydrotreating a hydrocarbon oil employing at least a first and a second reactor vessel, which process comprises:

(i) contacting the hydrocarbon oil in the first reactor vessel at elevated temperature and pressure with a hydrotreating catalyst in the presence of a hydrogen-containing gas, thereby consuming hydrogen;
(ii) separating the effluent of step (i) into partly hydrotreated hydrocarbon oil and contaminated hydrogen containing gas using a stripping column employing used hydrogen-containing gas as stripping gas;
(iii) contacting partly hydrotreated hydrocarbon oil obtained in step (ii) in the second reactor vessel at elevated temperature and pressure with a hydrotreating catalyst in the presence of clean hydrogen-containing gas, thereby consuming hydrogen, wherein at least 80% of the hydrogen consumed in steps (i) and (iii) is supplemented by additional clean hydrogen-containing gas fed to the second reactor;
(iv) separating the product from step (iii) in the second reactor vessel into a hydrotreated hydrocarbon oil and used hydrogen-containing gas, which hydrotreated hydrocarbon oil can be recovered as product and,
(v) transporting at least a portion of used hydrogen-containing gas obtained in step (iv) to step (ii) for use as stripping gas.

The process of the present invention uses effectively all gaseous components in the used hydrogen-containing gas in the stripping column. That entails that also gaseous hydrocarbons that may have been formed in the second hydrotreating reactor will be used in the stripping action. Moreover, since the used hydrogen-containing gas emerges directly from the second hydrotreating reactor, without any cooling, it may become available at hydrotreating conditions, which entails elevated temperature. The used hydrogen-containing gas at such elevated temperature will facilitate the stripping action further and will improve the heat recovery from the used hydrogen-containing gas. Preferably, the used hydrogen-containing gas has a temperature of at least 200° C., more preferably at least 250° C., even more preferably at least 300 and at most 400° C. The pressure of the used hydrogen containing gas is preferably at least 10 bar, more preferably at least 20 bar. The pressure of the used hydrogen containing gas is preferably at maximum 100 bar.

In the preferred embodiment that the hydrogen containing gas is cascaded from the second stage reactor to the interstage stripper to the first stage reactor, the gas flow is very effectively used, thereby minimizing the required compressor capacity. Further, since the hydrogen containing gas loop is not cooled, no let-down valves are needed, thereby minimizing the required compressor differential pressure.

In general it will be advantageous to treat a hydrocarbon oil of which a major amount, for example more than 70% by weight, suitably more than 80% by weight and preferably more than 90% by weight, is in the liquid phase at the process conditions prevailing in the first reactor vessel. Hydrocarbon oils that can suitably be hydrotreated according to the present invention are kerosene fractions, gas oil fractions and lubricating oils. Especially a gas oil fraction can very suitably be subjected to the present invention, as the environmental constraints on gas oils are tightening. A suitable gas oil would be one of which a major portion of the hydrocarbons, e.g. at least 75% by weight boils in the range of from 150 to 400° C. A suitable lubricating oil contains at least 95% by weight of hydrocarbons boiling in the range of from 320 to 600° C.

The hydrotreating process can be a hydrofinishing process in which the oil is marginally changed, it may be a hydrocracking process in which the average number of carbon atoms in the oil molecules is reduced, it may be a hydrodemetallisation process in which metal components are removed from the hydrocarbonaceous feedstock, it may be a hydrogenation process in which unsaturated hydrocarbons are hydrogenated and saturated, it may be a hydrodewaxing process in which straight chain molecules are isomerised, or it may be a hydrodesulphurisation process in which sulphur compounds are removed from the feedstock. It has been found that the present process is particularly useful when the hydrocarbonaceous feedstock comprises sulphur compounds and the hydrotreating conditions comprise hydrodesulphurisation conditions. The process is also very advantageous in the treatment of sulphur-containing feedstocks that contain so-called refractory sulphur compounds, i.e., dibenzothiophene compounds.

The hydrotreating conditions that can be applied in the process of the present invention are not critical and can be adjusted to the type of conversion to which the hydrocarbon oil is being subjected. Generally, the hydrotreating conditions comprise a temperature ranging from 250 to 480° C., preferably from 320 to 400° C., a pressure from 10 to 150 bar, preferably 20 to 90 bar, and a weight hourly space velocity of from 0.1 to 10 hr⁻¹, preferably from 0.4 to 4 hr⁻¹. The skilled person will be able to adapt the conditions in accordance with the type of feedstock and the desired hydrotreatment.

The catalyst used in the present process can also be selected in accordance with the desired conversion. Suitable catalysts comprise at least one Group VB, VIB and/or VIII metal of the Periodic Table of the Elements on a suitable carrier. Examples of suitable metals include cobalt, nickel, molybdenum and tungsten, but also noble metals may be used such as palladium or platinum. Especially when the hydrocarbon oil comprises sulphur, the catalyst suitably contains a carrier and at least one Group VIB and a Group VIII metal. Whereas these metals can be present in the form of their oxides, it is preferred to use the metals in the form of their sulphides. Since the catalyst may normally be produced in their oxidic form the catalysts may subsequently be subjected to a pre-sulphiding treatment which can be carried out ex situ, but is conducted preferably in-situ, in particular under circumstances that resemble the actual conversion.

The metals are suitably combined on a carrier. The carrier may be an amorphous refractory oxide, such as silica, alumina or silica alumina. Also other oxides, such as zirconia, titania or germania can be used. For hydrodewaxing processes, crystalline aluminosilicates, such as zeolite beta, ZSM-5, mordenite, ferrierite, ZSM-11, ZSM-12, ZSM-23 and other medium pore zeolites, can be used. When the hydrotreating conditions entail hydrocracking, the catalyst may advantageously comprise a different zeolite. Suitable zeolites are of the faujasite type, such as zeolite X or Y, in particular ultra-stable zeolite Y. Other, preferably large pore, zeolites are also possible. The zeolites are generally combined with an amorphous binder, such as alumina. The metals are suitably combined with the catalyst by impregnation, soaking, co-mulling, kneading or, additionally in the case of zeolites, by ion exchange. It is evident that the skilled person will know what catalysts are suitable and how such catalysts can be prepared.

By “clean hydrogen-containing gas” is understood a gas that contains less than 0.1% vol of hydrogen sulphide, based on the total volume of the gas, preferably less than 0.01% vol, more preferably less than 20 ppmv, and most preferably less than 5 ppmv. Examples of clean hydrogen-containing gas may include fresh make-up hydrogen, prepared by e.g., steam reforming, or a contaminated hydrogen-containing gas that has been subjected to a cleaning treatment, e.g., with an amine. Such contaminated gas may originate from the present process, but also contaminated hydrogen-containing gas from different sources or processes may be subjected to cleaning and subsequent use in the present process. The amount of hydrogen in clean hydrogen-containing gas is preferably at least 95% vol, more preferably at least 97% vol, based on the total clean hydrogen-containing gas.

In a first embodiment the hydrogen-containing gas that is used in step (i) in the first reactor is clean hydrogen-containing gas. This ensures that the amount of gas that needs to be fed into the first reactor can be minimised. Such gas may suitably be obtained from purification of contaminated hydrogen-containing gas, e.g., such contaminated gas that becomes available in the present process. The hydrogen-containing gas that is used in step (iii) in the second reactor is the clean hydrogen-containing gas required to replenish the hydrogen consumed in the first and second reactor, possibly supplemented with purified clean gas.

In a further preferred embodiment the effluent from the first reactor is passed to a gas-liquid separator before using the stripping column. The gaseous phase in the effluent typically contains large amounts, such as 0.5 to 5.0% vol, based on the total volume of the gaseous phase, of contaminants such as hydrogen sulphide and ammonia. This phase is therefore withdrawn as contaminated hydrogen-containing gas in the gas-liquid separator and may preferably be passed to a purification section, such as an amine scrubber. The liquid phase comprising partly hydrotreated hydrocarbon oil is withdrawn from the gas-liquid separator and passed to the stripping column. The stripping column is operated with used hydrogen-containing gas from the second reactor. The combination of used hydrogen-containing gas and stripped gas is preferably fed to the first reactor as hydrogen-containing gas. It is clear that in this embodiment the separation in step (ii) employs a gas-liquid separator in addition to a stripping column. The majority of the contaminants and lighter hydrocarbon components have been removed in the gas liquid separator. The residual contaminants that are fed to the first reactor represent a small amount and will not affect the hydrotreating process in the first reactor.

The first reactor in this preferred embodiment is operated with a hydrogen-containing gas that contains some contaminants.

Hydrogen is being consumed in the hydrotreatment steps. Generally, the hydrogen consumption for the hydrotreatment steps is not critical for the process and depends on the type of hydrocarbon oil that is being processed. Suitably, the hydrogen consumption in each of the reactors under hydrotreatment conditions ranges from 0.1 to 2.5% wt, based on the weight of the hydrocarbon oil for the first reactor and on the weight of the partly hydrotreated hydrocarbon oil for the second reactor. The hydrogen consumed in the first and second reactor is suitably being supplemented for at least 80% by addition of clean hydrogen-containing gas to the second reactor. In this way the amount of gas that gets contaminated with significant amounts of contaminants in the first reactor is minimised. Further minimisation can suitably be achieved by supplementing at least 90%, more preferably substantially 100% of the hydrogen consumed in the first and second reactor, with clean hydrogen-containing gas to the second reactor.

The effluent of the first reactor contains partly hydrotreated hydrocarbon oil. In the second step this partly hydrotreated hydrocarbon oil is separated from contaminated hydrogen-containing gas. In an advantageous embodiment the hydrocarbon oil to be treated is a gas oil that typically contains sulphur compounds. In the first reactor these sulphur compounds are converted to hydrogen sulphide, which contaminates the hydrogen-containing gas. In accordance with the process of the present invention the contaminated hydrogen-containing gas is separated from the partly hydrotreated hydrocarbon oil in a stripping column. In the stripping process used hydrogen-containing gas, recovered from step (iv), is being used as stripping gas. The contaminated hydrogen-containing gas thus obtained is suitably cleaned and used again as clean hydrogen-containing gas in step (iii), and optionally in step (i).

The treatment of contaminated hydrogen-containing gases, especially when contaminated with hydrogen sulphide and other sulphur compounds, such as carbon disulphide or carbon oxysulphide, is well known. A suitable way to remove these contaminants has been briefly described in EP-A 611 816, and is by amine scrubbing. Therefore, the contaminated hydrogen-containing gas is preferably cleaned by treating with an amine.

In such situations the contaminated hydrogen-containing gas is suitably contacted with an aqueous amine solution. The aqueous solution comprises one or more amine compounds. Suitable amine compounds are primary, secondary and tertiary amines. Preferably, the amines comprise at least one hydroxyalkyl moiety. The alkyl group in such moiety suitably comprises from 1 to 4 carbon atoms. In case of secondary and tertiary amines, the amine compounds preferably comprise one or more alkyl and hydroxyalkyl groups each with preferably from 1 to 4 carbon atoms. Suitable examples of amine compounds include monoethanol amine, monomethanol amine, monomethyl-ethanolamine, diethyl-monoethanolamine, diethanolamine, triethanolamine, di-isopropanolamine, diethyleneglycol monoamine, methyldiethanolamine and mixtures thereof. Other suitable compounds are N,N′-di(hydroxyalkyl)piperazine, N,N,N′,N′-tetrakis(hydroxyalkyl)-1,6-hexanediamine, in which the alkyl moiety may comprise from 1 to 4 carbon atoms.

The aqueous solution may also comprise physical solvents. Suitable physical solvents include tetramethylene sulphone (sulpholane) and derivatives, amides of aliphatic carboxylic acids, N-alkyl pyrrolidone, in particular N-methylpyrrolidine, N-alkyl piperidones, in particular N-methyl piperidone, methanol, ethanol, ethylene glycol, polyethylene glycols, mono- or di(C₁-C₄)alkyl ethers of ethylene glycol or polyethylene glycols, suitably having a molecular weight from 50 to 800, and mixtures thereof.

The concentration of the amine compound in the aqueous solution may vary within wide ranges. The skilled person will be able to determine suitable concentrations without undue burden. Advantageously, the aqueous solution comprises at least 15% wt of water, from 10 to 65% wt, preferably from 30 to 55% wt of amine compounds and from 0 to 40% wt of physical solvent, all percentages based on the weight of water, amine compound and physical solvent.

The conditions under which the contaminated hydrogen-containing gas is being treated with an amine suitably include a temperature of from 0 to 150° C., preferably, from 10 to 60° C., and a pressure of from 10 to 150 bar, preferably from 35 to 120 bar.

The stripping gas in the stripping column comprises used hydrogen-containing gas. Since the stripping gas becomes available from the hydrotreatment reaction in step (iii), it becomes available at elevated temperature. Since the elevated temperature has an improved stripping performance over the stripping performance of cool gas and counteracts the cooling effect of stripping, it is evidently clear that the present process provides an additional advantage in that an improved stripping action is being obtained. The used hydrogen-containing gas that is being used as stripping gas in step (ii) has advantageously a temperature of from 250 to 480° C., preferably from 320 to 400° C.

A portion of or the entire partly hydrotreated hydrocarbon oil is subjected to a further hydrotreatment in step (iii). As indicated above, the present process is especially advantageous when the hydrocarbon oil to be treated is a gas oil. Therefore, it is particularly preferred that the hydrotreating catalyst in step (i) is a hydrodesulphurisation catalyst and the hydrotreating catalyst in step (iii) is a hydrodewaxing catalyst or a hydrodearomatization catalyst. In such cases the hydrodesulphurization catalyst suitably comprises an optionally sulphided catalyst comprising one or more metals from Group V, VI and VIII of the Periodic Table of the Elements, on a solid carrier. As indicated earlier the solid carrier can be selected from any of the refractory oxides described above. The hydrodesulphurisation catalyst in particular may comprise one or more of the metals nickel and cobalt, and one or more of the metals molybdenum and tungsten. The catalyst may advantageously be sulphided as described above.

The hydrodewaxing catalyst suitably comprises as catalytically active metal one or more noble metals from Group VIII of the Periodic Table of the Elements on a solid carrier. Preferably the noble metal is selected from the group consisting of platinum, palladium, iridium and ruthenium. The carrier advantageously comprises a zeolite as described above in combination with a binder material. Suitable binder material includes alumina, silica and silica-alumina. However, other refractory oxides can also be used.

If the hydrotreating step in step (iii) is performed, using a hydrodewaxing catalyst or a hydrodearomatization catalyst, the conditions that can be applied in the process of the present invention comprise generally a temperature ranging from 200 to 400° C., preferably from 250 to 350° C., a pressure from 10 to 150 bar, preferably 20 to 90 bar, and a weight hourly space velocity of from 0.1 to 10 hr⁻¹, preferably from 0.4 to 4 hr⁻¹. The skilled person will be able to adapt the exact conditions in accordance with the type of feedstock.

In step (iv) the effluent of the hydrotreatment in the second reactor is recovered and separated into a hydrotreated hydrocarbon oil and used hydrogen-containing gas. In accordance with step (v) of the present process at least a portion of the used hydrogen-containing gas is transferred to step (ii) for use as stripping gas. Preferably, at least 90% vol of the used hydrogen-containing gas is transferred to step (ii), more preferably at least 95% vol, and most preferably, the entire volume of used hydrogen-containing gas is transferred to step (ii).

The separation in step (iv) can be carried out in any suitable way. A suitable method involves the use of separation means inside the second reactor comprising a downwardly extending plate having an opening between the lower edge of the plate and the wall of the reactor vessel. Preferably, a downwardly extending flange has been provided at the lower edge of the plate. This is in accordance with a similar plate that has been described in EP-A 611 861. Alternatively, one or more different separation trays can be used in the lower part of the second reactor vessel. In a further embodiment, the separation of the effluent of the hydrotreatment in the second reactor is performed in a separate gas-liquid separator, optionally with additional heat integration. The effluent, before or after separation, can suitably be used for heat exchange with the partly hydrotreated hydrocarbon oil emerging from the stripping column. This has the advantage that the effluent is cooled whilst the partly hydrotreated hydrocarbon oil can be heated to the desired hydrotreating temperature without the use of an external heat supply, such as an additional furnace. It will be evident that such represents a considerable economical and heat-efficient advantage.

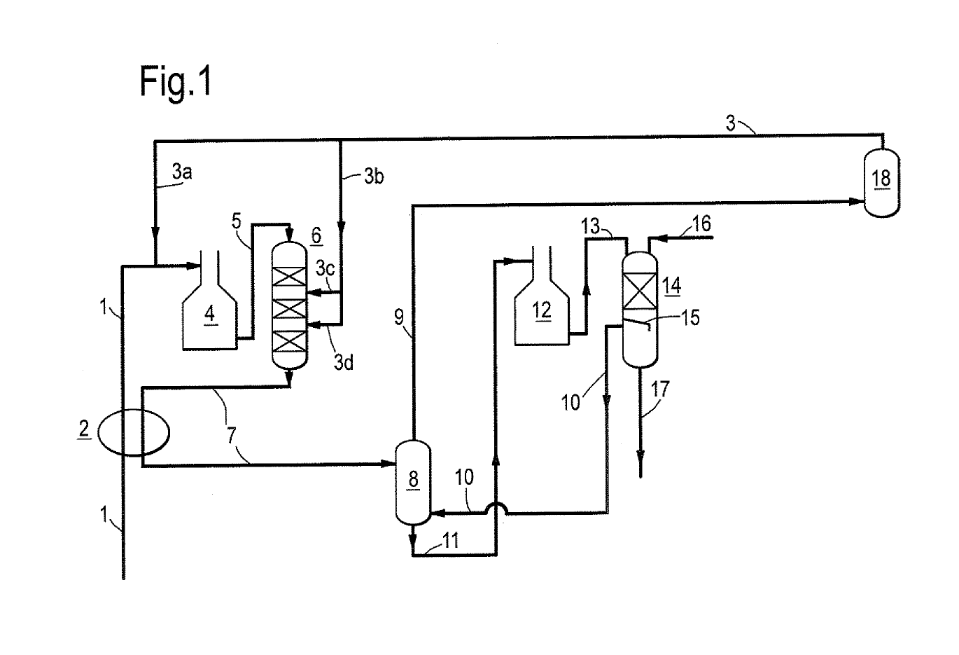

FIG. 1 shows a simplified flow scheme of an embodiment of the present invention.

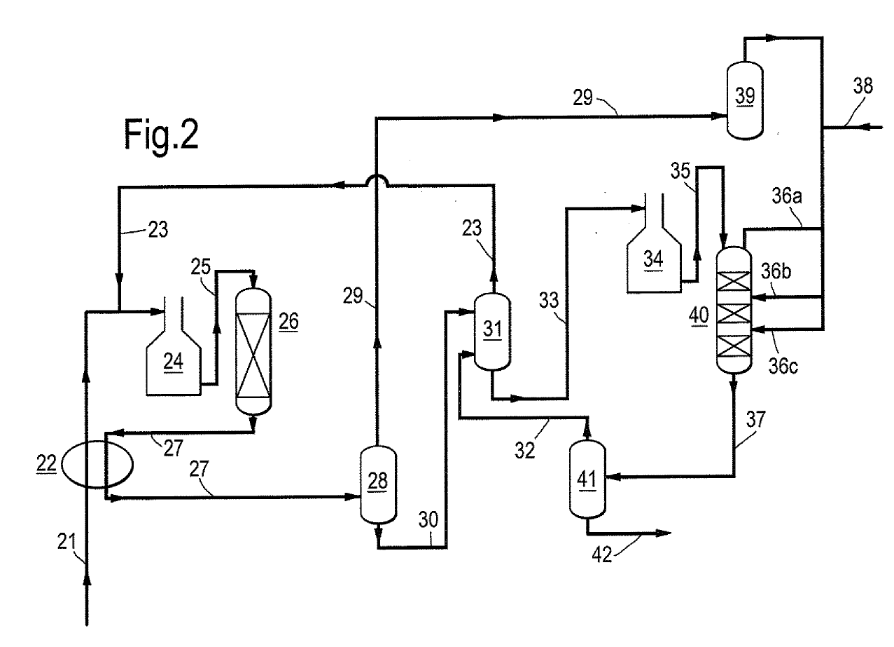

FIG. 2 shows an alternative embodiment of the present process.

FIG. 1 shows a line 1 via which a hydrocarbon oil is passed trough a heat exchanger 2 and to which clean hydrogen-containing gas is added via a line 3a. The combination of hydrogen-containing gas and hydrocarbon oil is passed through a furnace 4 and the heated combination is passed via a line 5 to a first hydrotreating reactor 6. The first hydrotreating reactor 6 has been provided with three catalyst beds. However, the number of catalyst beds is not critical and van be adjusted to meet the required hydrotreating conditions. Between two subsequent beds clean hydrogen-containing gas is added via lines 3c and 3d, respectively. In principle, the flow in the first and second reactor can be upwards or downwards. It is preferred to pass the hydrogen-containing gases and hydrocarbon oil or partly hydrotreated hydrocarbon oil concurrently through the reactor vessels in a downflow direction. In this way the gas flow and the liquid flow can be controlled in a reliable way. Further, reaction temperatures may be more easily controlled. The effluent from the first reactor is withdrawn via a line 7. The effluent is also passed through heat exchanger 2 to preheat the hydrocarbon oil to be treated, and subsequently passed to a stripping column 8. In the stripping column stripping gas in the form of used hydrogen-containing gas is fed into the lower part via a line 10 and the gaseous components in the effluent from line 7 together with the stripping gas are withdrawn as contaminated hydrogen-containing gas via a line 9. The contaminated hydrogen-containing gas is treated in an amine absorption column 18 and purified, clean hydrogen-containing gas is recovered via a line 3. The line 3 is split into the line 3a that leads hydrogen-containing gas to the hydrocarbon oil and a line 3b that splits subsequently into lines 3c and 3d to provide the first reactor 6 with additional hydrogen for reactor temperature control. It is appreciated that whereas the amine absorption is shown in the Figure as a single absorption column 18 the amine treatment unit comprises absorption and desorption columns and, optionally, one or more compressors. Further, the clean hydrogen-containing gas in the line 3 may be subjected to heat exchange with one or more other process streams, such as the contaminated hydrogen-containing gas in the line 9 and/or the effluent from the first reactor in the line 7. Stripped, partly hydrotreated hydrocarbon oil is discharged from the stripping column 8 via a line 11. If desired, the partly hydrotreated hydrocarbon oil in the line 11 is passed through a furnace 12, and the heated oil is passed via a line 13 into a second reactor 14. Clean hydrogen-containing gas, in this particular case fresh make-up hydrogen, is passed into the reactor 14 via a line 16. In accordance with the present invention at least 80% of the hydrogen that needs to be added, because it was consumed in reactors 6 and 14, will be added to reactor 14. It will be evident to the skilled person, that, if desired, a portion of fresh make-up hydrogen, i.e. up to 20% of the hydrogen consumed, can be supplemented with a stream of hydrogen-containing gas from line 3. The upper part of the reactor 14 is provided with a catalyst bed whereas the lower part has been provided with a separation tray 15 which allows the reaction product from the catalyst bed to flow into the lower portion of the reactor, but prohibits the backflow for gaseous components. The reaction product is being separated into a hydrotreated hydrocarbon oil and used hydrogen-containing gas. The gaseous components, i.e. used hydrogen-containing gas, is withdrawn from the reactor 14 via the line 10, which passes the used hydrogen-containing gas to the stripping column 8. Liquid hydrotreated hydrocarbon oil is recovered via a line 17. The products in line 17 may be fractionated in any known manner.

FIG. 2 shows a simplified flow scheme of an alternative embodiment. It shows a line 21 through which a hydrocarbon oil is passed through a heat exchanger 22 and to which a hydrogen-containing gas is added via a line 23. The hydrogen-containing gas in line 23 comes from a stripping column 31 and comprises hydrogen that has been in contact with a hydrotreating catalyst in a reactor 40 and the stripping column 31. The combined hydrogen-containing gas and hydrocarbon oil is heated in a furnace 24 and via a line 25 passed to a first hydrotreating reactor 26. The effluent of the reactor 26 is passed via the heat exchanger 22 in order to preheat the hydrocarbon oil, to a gas-liquid separator 28. The liquid product, containing partly hydrotreated hydrocarbon oil, is passed to the stripping column 31 via a line 30, and the gaseous product, containing a significant portion of contaminants, viz., the contaminants that were present in the hydrogen-containing gas plus those that were formed in the reaction in the reactor 26, is withdrawn from the gas-liquid separator via a line 29. The partly hydrotreated hydrocarbon oil in the stripping column 31 is subjected to stripping with used hydrogen-containing gas that stems from the second hydrotreating reactor 40. The stripping gas with any volatile compound that is withdrawn from the partly hydrotreated hydrocarbon oil is discharged via the line 23. The gas in line 23 will contain hydrogen and some light gaseous hydrocarbons and only a small portion of remaining heteroatoms-containing contaminants, such as hydrogen sulphide and ammonia. This gas is used as hydrogen-containing gas for the first reactor. Since the majority of the contaminants have been removed in the gas liquid separator 28 via line 29, the gas in line 23 can adequately be used as hydrogen-containing gas for the hydrotreatment in reactor 26. To the extent needed, the partly hydrotreated hydrocarbon oil obtained in the stripping column 31 can be passed therefrom via a line 33 to a furnace 34 where it is heated to the desired temperature for the second reactor. With proper heat integration between the second reactor effluent and the partly hydrotreated hydrocarbon oil that is used as feed to the second reactor, the furnace 34 may be omitted allowing the second reactor to operate in a so-called ‘autothermal’ mode. The heated oil is passed via a line 35 into the second reactor 40, where it is combined with clean hydrogen-containing gas supplied via a line 36a. Between subsequent catalyst beds additional hydrogen may be supplied via line 36b and 36c, respectively. Via a line 37 the reaction product of the reactor 40 is passed to a hot gas-liquid separator 41 in which hydrotreated hydrocarbon oil is separated from used hydrogen-containing gas. The used hydrogen-containing gas is removed via line 32 and passed to the stripping column 31. The hydrotreated hydrocarbon oil is discharged via a line 42 and recovered as product. The product in the line 42 may be subjected to fractionation to obtain the desired specified hydrocarbon product.

The contaminated hydrogen-containing gas in line 29 is passed to an amine treating unit, here represented by a column 39. In the column 39 contaminants are removed from the contaminated hydrogen-containing gas, resulting in clean hydrogen-containing gas that is withdrawn via a line 36. Fresh make-up hydrogen, in this case in an amount to supplement 100% of the hydrogen that is being consumed in the process, is added to the clean hydrogen-containing gas in the line 36 via a line 38. The line 36 may split into the lines 36a, 36b and 36c for supplying hydrogen to the reactor 40 at different locations.

It will be appreciated that the Figures do not show auxiliary equipment that is usually present, such as valves, pumps, compressors, expanders, control equipment etc. The skilled person will understand where this auxiliary equipment is desired.

1. A process for hydrotreating a hydrocarbon oil employing at least a first and a second reactor vessel, which process comprises:
(i) contacting the hydrocarbon oil in the first reactor vessel at elevated temperature and pressure with a hydrotreating catalyst in the presence of a hydrogen-containing gas, thereby consuming hydrogen;
(ii) separating the effluent of step (i) into partly hydrotreated hydrocarbon oil and contaminated hydrogen-containing gas using a stripping column employing used hydrogen-containing gas as stripping gas;
(iii) contacting partly hydrotreated hydrocarbon oil obtained in step (ii) in the second reactor vessel at elevated temperature and pressure with a hydrotreating catalyst in the presence of clean hydrogen-containing gas, thereby consuming hydrogen, wherein at least 80% of the hydrogen consumed in steps (i) and (iii) is supplemented by additional clean hydrogen-containing gas fed to the second reactor;
(iv) separating the product from step (iii) in the second reactor vessel into a hydrotreated hydrocarbon oil and used hydrogen-containing gas, which hydrotreated hydrocarbon oil can be recovered as product and,
(v) transporting at least a portion of used hydrogen-containing gas obtained in step (iv) to step (ii) for use as stripping gas.

2. A process according to claim 1, in which the used hydrogen-containing gas has a temperature of at least 200° C.

3. A process according to claim 1, in which the used hydrogen-containing gas has a pressure of at least 10 bar.

4. A process according to claim 1, in which the hydrocarbon oil to be hydrotreated is a gas oil which contains at least 75% by weight of hydrocarbons boiling in the range of from 150 to 400° C.

5. A process according to claim 1, in which the hydrocarbon oil to be hydrotreated is a lubricating oil which contains at least 95% by weight of hydrocarbons boiling in the range of from 320 to 600° C.

6. A process according to claim 1, in which the hydrotreating conditions comprise a temperature ranging from 250 to 480° C., a pressure from 10 to 150 bar, and a weight hourly space velocity of from 0.1 to 10 hr⁻¹.

7. A process according to claim 1, in which the clean hydrogen-containing gas contains less than 0.1% by volume of hydrogen sulphide.

8. A process according to claim 1, in which the effluent from the first reactor is passed to a gas-liquid separator before using the stripping column.

9. A process according to claim 1, in which contaminated hydrogen-containing gas obtained in step (ii) is cleaned and used again in step (iii), and optionally in step (i).

10. A process according to claim 9, in which the contaminated hydrogen-containing gas is cleaned by treating with an amine.

11. A process according to claim 1, in which at least 90% of the hydrogen consumed in steps (i) and (iii) is supplemented by additional clean hydrogen-containing gas fed to the second reactor.

12. A process according to claim 1, in which the used hydrogen-containing gas that is being used as stripping gas in step (ii) has a temperature of from 250 to 480° C.

13. A process according to claim 1, in which the hydrotreating catalyst of step (i) is a hydrodesulphurization catalyst and the hydrotreating catalyst of step (iii) is a hydrodewaxing catalyst.

14. A process according to claim 13, in which the hydrodesulphurization catalyst comprises one or more metals from Group VB, VIB and VIII of the Periodic Table of the Elements, on a solid carrier.

15. A process according to claim 14, in which the hydrodesulphurization catalyst comprises one or more of the metals cobalt and nickel, and one or more of the metals molybdenum and tungsten.

16. A process according to claim 13, in which the hydrodewaxing catalyst used in step (iii) comprises as catalytically active metal one or more noble metals from Group VIII of the Periodic Table of the Elements on a solid carrier.

17. A process according to claim 16, in which the noble metals are selected from the group consisting of platinum, palladium, iridium and ruthenium.
